(19) United States
(12) Patent Application Publication
(10) Pub. No.: US 2025/0186731 A1
(43) Pub. Date: Jun. 12, 2025

(54) Oxygen Treatment Device for Mammals

(71) Applicant: Ergo-Flex Technologies, LLC, Conroe, TX (US)

(72) Inventor: J.T. Anderson, Conroe, TX (US)

(73) Assignee: Ergo-Flex Technologies, LLC, Conroe, TX (US)

(21) Appl. No.: 18/979,464

(22) Filed: Dec. 12, 2024

Related U.S. Application Data
(63) Continuation-in-part of application No. 18/819,520, filed on Aug. 29, 2024.
Application No. 18/819,520 is a continuation of application No. 17/151,515, filed on Jan. 18, 2021, now Pat. No. 12,083,283.
Application No. 17/151,515 is a continuation of application No. 15/914,888, filed on Mar. 7, 2018, now Pat. No. 10,894,139.
(60) Provisional application No. 62/619,387, filed on Jan. 19, 2018.

(51) Int. Cl. A61M 16/10; A61B 5/00; A61B 5/021; A61B 5/024; A61B 5/08; A61B 5/11; A61B 5/145; A61M 16/00; A61M 16/06; A61M 16/12; A63B 23/18

(52) U.S. Cl. CPC A61M 16/101; A61B 5/0022; A61M 16/12; A63B 23/18; A61B 5/021; A61B 5/024; A61B 5/0816; A61B 5/1118; A61B 5/14542; A61B 5/4836; A61B 5/4839; A61B 5/4866; A61B 2503/10; A61B 2505/09; A61M 2016/003; A61M 16/024; A61M 16/06; A61M 2016/103; A61M 2202/0208; A61M 2205/52; A61M 2230/06; A61M 2230/30; A61M 2230/50

(57) ABSTRACT

A method and apparatus vary the oxygen concentration level of air delivered to a person for treatment on training purposes. The apparatus includes a display device for displaying current physiological data as well as data from previous sessions for comparison purpose. The oxygen level is precisely controlled by a central processing unit in response to input data from the person.

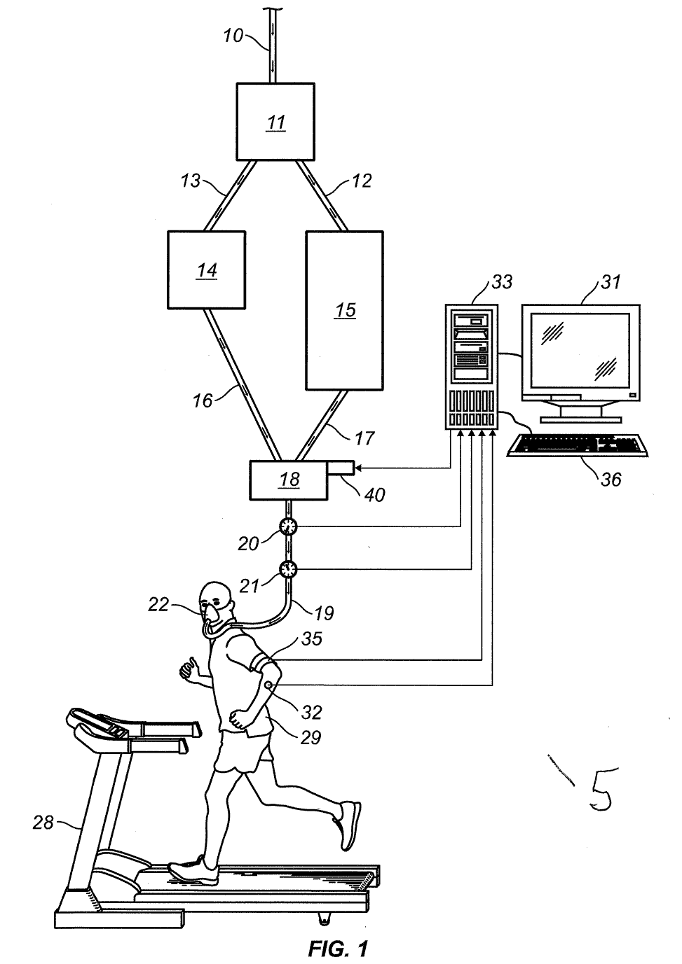

This application is continuation-in-part of U.S. patent application Ser. No. 18/819,520 filed Aug. 29, 2024, which is a continuation of U.S. patent application Ser. No. 17/151,515 filed Jan. 18, 2021 and titled “Oxygen Treatment Device for Mammals”, which is a continuation of U.S. patent application Ser. No. 15/914,888 filed Mar. 7, 2018 and issued as U.S. Pat. No. 10,894,139 on Jan. 19, 2021, titled “Oxygen Treatment Device for Mammals”, which claims priority to provisional application Ser. No. 62/619,387 filed Jan. 19, 2018, titled “Oxygen Treatment Device for Mammals”, the entire content of which is incorporated herein by reference thereto.

### BACKGROUND OF INVENTION

### Field of the Invention

This invention relates to the field of oxygen therapy, and more specifically to processes and devices to provide oxygen therapy and related protocols to improve health and repair damage for a user.

### Background

Systems for delivering varying concentrations of oxygen to people for training purposes are known. However, drawbacks to such systems include that they are not designed to provide precise concentrations of oxygen. Further drawbacks include that they do not include sufficient monitoring or measuring devices for the physiological measurements so that instantaneous information (such as heat rate, blood oxygen level, blood pressure as well as data related to the improvement of blood oxygen levels and other data compared to previous exercising sessions) can be calculated and displayed on a monitor. Additional drawbacks include that the systems are not designed in one system to provide numerous health benefits such as anti-aging, weight loss, brain health, and the like.

Consequently, there is a need for improved processes and devices adapted to supply a precise concentration of oxygen in air to a person for training or health reasons. Further needs include improved processes and devices for improving overall health and repairing damage to a person.

### BRIEF SUMMARY OF SOME OF THE PREFERRED EMBODIMENTS

These and other needs in the art addressed in an embodiment by an oxygen therapy system. The oxygen therapy system includes an oxygen concentrator and a valve. Concentrated oxygen and exhaust air from the oxygen concentrator are directed to the valve and exit the valve as treatment air. The treatment air is provided to a user. The oxygen therapy system also includes a breathing mask. The treatment air is provided to the user via the breathing mask. In addition, the oxygen therapy system includes an oxygen therapy protocol. The treatment air is provided to the user according to the oxygen therapy protocol. The oxygen therapy protocol includes a hypoxic state and a hyperoxic state. The hyperoxic state follows the hypoxic state. The oxygen therapy protocol has an oxygen therapy protocol duration from about 10 minutes to about 30 minutes. The hypoxic state has a duration from about 2 minutes to about 4 minutes. The hyperoxic state has a duration from about 1 minute to about 3 minutes. A hypoxic state oxygen concentration of the treatment air in the hypoxic state is from about 10% to about 15%. A hyperoxic state oxygen concentration of the treatment air in the hyperoxic state is from about 30% to about 80%. The oxygen therapy protocol includes more than one hypoxic state and more than one hyperoxic state. Each of the hypoxic states are followed by each of the hyperoxic states in a successive order with a hyperoxic state that follows a hypoxic state in turn followed by a succeeding hypoxic state. The successive order is carried out until the oxygen therapy protocol duration has been achieved. The oxygen therapy protocol includes a metabolic disorder protocol. The oxygen therapy protocol includes a mitochondrial protocol. The oxygen therapy protocol includes a weight loss protocol. The oxygen therapy protocol includes a neurological protocol. The oxygen therapy protocol includes a cardiovascular protocol. The oxygen therapy protocol includes an athletic performance protocol. The oxygen therapy protocol includes a respiratory protocol. The oxygen therapy protocol includes a lactate clearance protocol.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWING

The figure is a schematic drawing showing the components of an apparatus for carrying out an embodiment of an oxygen therapy system.

### DETAILED DESCRIPTION OF THE INVENTION

Embodiments of oxygen therapy system 5 are shown in the figure. As shown in the figure, embodiments of oxygen therapy system 5 include ambient air entering oxygen concentrator 11 via conduit 10. Oxygen concentrator 11 may be any device suitable for concentrating oxygen from ambient air. From the ambient air, oxygen concentrator 11 provides concentrated oxygen and exhaust air. Concentrated oxygen from oxygen concentrator 11 enters closed reservoir 15 via conduit 12, and the exhaust air from oxygen concentrator 11 is provided to closed reservoir 14 via conduit 13. It is to be understood that the exhaust air has a lower concentration of oxygen than that of the ambient air, and concentrated oxygen has a higher concentration of oxygen than ambient air.

Closed reservoir 14 and closed reservoir 15 are connected to valve 18 via conduit 16 and conduit 17. Valve 18 may be controlled by any suitable means. In an embodiment, valve 18 is controlled via actuator 40 by a central processing unit 33 to control the oxygen level of the treatment air leaving valve 18. In an embodiment, the treatment air leaving valve 18 is a mixture of concentrated oxygen and the exhaust air produced from oxygen concentrator 11. The treatment air from valve 18 is conveyed to user 29 via conduit 19. The treatment air may be conveyed to user 29 by any suitable means for user 29 to have the air enter the body of user 29. In an embodiment as shown, the treatment air is conveyed to user 29 by breathing mask 22. Flow meter 20 and an oxygen concentration meter 21 may be located within conduit 19. User 29 is thus supplied treatment air, the oxygen content of which may be varied.

In an embodiment, user 29 is subjected to physical activity on exercise device 28. Exercise device 28 may be any device by which user 29 may carry out a cardiovascular activity. Without limitation, exercise device 28 may be an exercise bike, a treadmill (as illustrated), a shaker plate, and the like. In an embodiment, exercise device 28 is an exercise bike. The subject's physical characteristics may be measured. Such physical characteristics include, without limitation, blood pressure, heart rate, blood oxygen level, temperature, or any combinations thereof. Such physical characteristics may be measured by any suitable means. In an embodiment, such physical characteristics are measured by one or more sensors. The sensors may be any sensors suitable for measuring such physical characteristics. In embodiments as shown, oxygen therapy system 5 has sensors 32, 35. In an embodiment, sensors 32, 35 measure physical characteristics, and the measured physical characteristics (i.e., measured physical characteristics data) are sent to central processing unit 33. Central processing unit 33 processes the data and can vary the oxygen concentration of the treatment air supplied to user 29 by wireless manipulation of valve 18 by actuator 40 according to algorithms stored in central processing unit 33. In embodiments, valve 18 may be manually manipulated to obtain the desired oxygen concentration in the treatment air.

As further shown, embodiments of oxygen therapy system 5 include visual displays whereby real-time and past-time information are visually available. For instance, real-time and past-time information are available to user 29. In embodiments, the real-time information and past-time information includes physical characteristics and any other suitable information on user 29. In embodiments as shown, oxygen therapy system 5 includes display device 31 that displays real-time information, past-time information, or any combinations thereof. In some embodiments, display device 31 is connected to central processing unit 33 so that information (i.e., real-time information) is visible to user 29. A keyboard 36 for data input may also be provided. However, it is to be understood that data may be input by any suitable means. Any desirable information may be input into central processing unit 33. Such information may include sex, height, weight, age, medical history, or any combination thereof of user 29.

In an embodiment, oxygen therapy system 5 may be used to train user 29 for higher altitudes. In embodiments, valve 18 may be set to deliver an oxygen concentration level of about 21%, which is normal. User 29 may exercise to raise user's 29 heart beat rate to a target level. At that point when the target level is achieved, the oxygen concentration of treatment air is fed to user 29 at the desired elevation, which will be lower than 21% to simulate the less dense air at higher elevations. For example, at 6,000 ft, the effective oxygen level may be about 16.6%. The effect of lowering the oxygen concentration on the subject's heartbeat, blood pressure and blood oxygen level may be monitored by sensors 32, 35 and stored in central processing unit 33. After a given period of time, the oxygen concentration may be raised to 21%, and the time it takes for the heart rate of user 29 to return to the baseline level may be measured as well as the oxygen blood level with sensors 32, 35. Subsequent tests may be performed to measure user's 29 improvement in recovery time and also in blood oxygen levels, as well as user's 29 endurance. All this information may be displayed on display device 31 in real-time and may be compared to prior tests. In embodiments, central processing unit 33 compare the real-time information with prior tests.

In other embodiments of oxygen therapy system 5, another use is to supply treatment air with increased oxygen levels to user 29 in order to raise the oxygen levels in the blood of user 29. Without limitation, repeated treatments have resulted in removing toxins from the blood and increasing blood oxygen levels. Further, without limitation, these results may have a positive effect on the functioning of the organs of user 29 and an overall improvement in the health of user 29.

In embodiments, closed reservoir 14 and closed reservoir 15 may be any suitable closed reservoir for use with oxygen therapy system 5. In an embodiment, close reservoir 14 and close reservoir 15 may be fabricated using gas impermeable fabric or cloth and may be separate from each other or attached at a common point.

In some embodiments, display device 31 may include more than one screen. For instance, each screen may display different parameters concurrently.

In an embodiment, sensors 32, 35, flow meter 20, and/or oxygen concentration meter 21 may communicate with central processing unit 33 wirelessly and vice versa.

In embodiments shown of oxygen therapy system 5, oxygen therapy system includes responsive oxygen therapy, exercise with oxygen therapy, or combinations thereof. Responsive oxygen therapy refers to adjusting the amount of oxygen delivered to user 29 based on real-time needs by monitoring user's 29 blood oxygen levels and automatically increasing or decreasing the oxygen flow to user 29 accordingly based on a desired oxygen therapy protocol. Responsive oxygen therapy may be carried out with user 29 exercising on exercise device 28 or in a stationary position (i.e., sitting, standing, or laying down) without exercising. Exercise with oxygen therapy refers to user 29 breathing increased concentrations of oxygen while exercising. In embodiments, oxygen therapy system 5 is not based on a fixed oxygen delivery rate. Without limitation, responsive oxygen therapy comprises intermittent hypoxia-hyperoxia training (IHHT). In embodiments, responsive oxygen therapy uses alternating low-oxygen (i.e., hypoxia) and high-oxygen (i.e., hyperoxia) conditions for user 29. Without being limited by theory, such alternating hypoxia and hyperoxia states may stimulate user's 29 body's response to stress and optimize cellular function. Further, without being limited by theory, such response may activate user's 29 sympathetic nervous system to facilitate the cells' adaptation, recovery, and strength. The hypoxia state may facilitate production of new blood vessels and stimulate mitochondria to function more efficiently. In the hypoxic state, oxygen levels are typically reduced. In embodiments, oxygen levels are reduced to mimic altitudes of 3,000 to 5,000 meters. At these levels, user's 29 body may move to anaerobic metabolism, which may increase reliance on glycogen as the primary fuel source. With repeated exposure to the reduced oxygen levels, user's body 29 may adapt by enhancing uptake efficiency and increasing the production of erythropoietin, which increase in red blood cells may improve the oxygen-carrying capacity of the blood. The hyperoxic state increases oxygen in the blood, which may increase energy, reduce inflammation, and promote cellular regeneration. The hyperoxic state may have 30% to 40% oxygen in some embodiments. In embodiments, oxygen therapy system 5 combines responsive oxygen therapy with exercise with oxygen therapy. Oxygen therapy system 5 does not have user 29 enclosed for the oxygen therapy protocols. For instance, user 29 is not disposed in a chamber such as a hyperbaric chamber. Oxygen therapy system 5 has a hypoxic state followed by a hyperoxic state which is followed by another hypoxic state which is then followed by another hyperoxic state and so forth accordingly until the desired oxygen therapy protocol duration has been achieved.

In embodiments, oxygen therapy system 5 has oxygen therapy protocols. The oxygen therapy protocols have varying durations in total treatment time, varying durations in rotations between hypoxic and hyperoxic states, varying percent oxygen concentrations for hypoxic and hyperoxic states. The oxygen therapy protocols cycle between hypoxic states and hyperoxic states depending on the treatment desired and the desired result. Oxygen therapy protocols may be for any desired treatment and result. In embodiments, the oxygen therapy protocols include metabolic disorder protocols, mitochondrial protocols, weight loss protocols, neurological protocols, cardiovascular protocols, athletic performance protocols, respiratory protocols, lactate clearance protocols, or any combinations thereof.

In an embodiment, oxygen therapy system 5 has oxygen therapy protocols that last about 10 minutes to about 30 minutes, alternatively about 20 minutes to about 30 minutes, and alternatively about 20 minutes to about 25 minutes, further alternatively about 15 minutes to about 20 minutes, alternatively from about 10 minutes to about 25 minutes, alternatively from about 10 minutes to about 20 minutes, alternatively from about 10 minutes to about 15 minutes, and alternatively from about 15 minutes to about 30 minutes, and further about 10 minutes.

In an embodiment, oxygen therapy system 5 has oxygen therapy protocols with the hypoxic state having a duration of about 2 minutes to about 4 minutes and the hyperoxic state having a duration of about 1 minute to about 3 minutes, alternatively the hypoxic state having a duration of about 4 minutes and the hyperoxic state having a duration of about 1 minute, further alternatively the hypoxic state having a duration of about 2 minutes and the hyperoxic state having a duration of about 3 minutes, and alternatively the hypoxic state having a duration of about 3 minutes and the hyperoxic state having a duration of about 2 minutes.

In embodiments, oxygen therapy system 5 has oxygen concentrations provided to user 29 in the hypoxic state from about 10% to about 15% and in the hyperoxic state from about 70% to about 80%, alternatively in the hypoxic state from about 12% to about 15% and in the hyperoxic state from about 30% to about 40%, further alternatively in the hypoxic state from about 14% to about 15% and in the hyperoxic state from about 35% to about 40%, and further alternatively in the hypoxic state from about 14% to about 15% and in the hyperoxic state from about 30% to about 40%, and alternatively in the hypoxic state of about 15% and in the hyperoxic state from about 30% to about 35%.

Embodiments of oxygen therapy system 5 include metabolic disorder protocols. Without limitation, metabolic disorder protocols enhance metabolic flexibility and fat oxidation to support weight management and insulin sensitivity. In an embodiment, the metabolic disorder protocol is of a duration from about 20 minutes to about 30 minutes, alternatively from about 20 minutes to about 25 minutes. The metabolic disorder protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of 3 minutes of a hypoxic state followed by 2 minutes of a hyperoxic state, of 4 minutes of a hypoxic state followed by 2 minutes of a hyperoxic state, of 3 minutes of a hypoxic state followed by 4 minutes of a hyperoxic state. The metabolic disorder protocol has a hypoxic oxygen concentration from about 10% to 15%, alternatively from about 12% to about 15%, and alternatively from about 10% to about 12% and has a hyperoxic state oxygen concentration from about 30% to about 40%, alternatively from about 30% to about 70%, alternatively from about 30% to about 80%, and alternatively from about 40% to about 70%, and alternatively from about 40% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic stage involves increased oxygen levels for user 29. In embodiments, the metabolic disorder protocols are carried out 3-5 times a week until obtaining the desired result. They hypoxic state may stimulate metabolic recovery, enhance fat oxidation, support weight management and metabolic health. The hyperoxic state which may be shorter than the hypoxic state may facilitate recovery, reduce oxidative stress, and maintain metabolic adaptations from hypoxia.

Embodiments of oxygen therapy system 5 include mitochondrial health protocols. Without limitation, mitochondrial health protocols support cellular resilience, mitochondrial health, and reduced oxidative stress for anti-aging. In an embodiment, the mitochondrial health protocol is of a duration from about 15 minutes to about 30 minutes, alternatively from about 15 minutes to about 20 minutes, and alternatively from about 20 minutes to about 30 minutes. The mitochondrial health protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of 2 minutes of a hypoxic state followed by 3 minutes of a hyperoxia state. The mitochondrial health protocol has a hypoxic state oxygen concentration from about 10% to 15%, alternatively from about 10% to about 14%, and alternatively from about 10% to about 12% and further alternatively from about 14% to about 15%, and has a hyperoxia state oxygen concentration from about 30% to about 35%, alternatively from about 30% to about 80%, and alternatively from about 30% to about 70%, and from about 35% to about 70%, and from about 35% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the mitochondrial health protocol is carried out 3-5 times a week until obtaining the desired result, alternatively 3 times a week. The hypoxic state may stimulate mitochondrial efficiency and biogenesis, which may enhance cellular energy and longevity. The hyperoxic state may facilitate recovery, reduce oxidative stress, and maintain metabolic adaptations from hypoxia.

Embodiments of oxygen therapy system 5 include weight loss protocols. Without limitation, weight loss protocols optimize fat oxidation and metabolic efficiency to support sustainable weight loss, which includes a gradual depletion of glycogen stores and a shift towards fat oxidation. Further without being limited by theory, the prolonged hypoxic state may enhance this shift. In an embodiment, the weight loss protocol is of a duration from about 20 minutes to about 30 minutes, alternatively from about 20 minutes to about 25 minutes, and alternatively from about 25 minutes to about 30 minutes. The weight loss protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of 4 minutes of a hypoxic state followed by 1 minute of a hyperoxic state. The weight loss protocol has a hypoxic state oxygen concentration from about 10% to 15%, alternatively from about 12% to about 15%, from about 10% to about 12%, and has a hyperoxic state oxygen concentration from about 30% to about 40%, alternatively from about 30% to about 80%, and alternatively from about 30% to about 70%, and from about 40% to about 70%, and from about 40% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the weight loss protocols are carried out 3-5 times a week until obtaining the desired result, alternatively 3 times a week.

Embodiments of oxygen therapy system 5 include neurological protocols. Without limitation, neurological protocols support neuroplasticity, cognitive resilience, and recovery in neurological conditions. In an embodiment, the neurological protocol is of a duration from about 15 minutes to about 30 minutes, alternatively from about 15 minutes to about 20 minutes, and alternatively from about 20 minutes to about 30 minutes. The neurological protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of about 2 minutes of a hypoxic state followed by about 3 minutes to about 4 minutes of a hyperoxic state, alternatively about 3 minutes of a hyperoxic state. The neurological protocol has a hypoxic state oxygen concentration from about 10% to 15%, alternatively from about 12% to about 15%, and alternatively from about 10% to about 12% and further alternatively about 15%, and has a hyperoxic state oxygen concentration from about 30% to about 40%, alternatively from about 30% to about 80%, and alternatively from about 30% to about 70%, and from about 40% to about 70%, and from about 40% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic stage involves increased oxygen levels for user 29. In embodiments, the neurological protocols are carried out 2-3 times a week until obtaining the desired result. The hypoxic state may initiate neuroprotective pathways by activating hypoxia-inducible factors, which may stimulate angiogenesis and neurogenesis that may increase blood supply to the brain. The hyperoxic state enhance oxygen supply to brain tissue, promote mitochondrial repair, and support overall neuronal recovery.

Embodiments of oxygen therapy system 5 include cardiovascular protocols. Without limitation, cardiovascular protocols improve vascular flexibility, circulation, oxygen efficiency, and may support impaired nerve function for overall wellness. In an embodiment, the cardiovascular protocol is of a duration from about 15 minutes to about 30 minutes, alternatively from about 15 minutes to about 20 minutes, and alternatively from about 20 minutes to about 30 minutes. The cardiovascular protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of about 3 minutes of a hypoxic state followed by about 2 to about 3 minutes of a hyperoxic state, alternatively about 2 minutes of a hyperoxic state. The cardiovascular protocol has a hypoxic state oxygen concentration from about 10% to 15%, alternatively from about 12% to about 15%, and alternatively from about 10% to about 12% and further alternatively about 15%, and has a hyperoxic state oxygen concentration from about 30% to about 35%, alternatively from about 30% to about 80%, and alternatively from about 30% to about 70%, and from about 35% to about 70%, and from about 35% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the cardiovascular protocols are carried out 3 times a week until obtaining the desired result. The hypoxic state may by the reduced oxygen levels induce vascular flexibility, which may aid in the release of nitric oxide and may improve circulation. The hyperoxic state may provide an oxygen-rich environment to support the heart and blood vessels.

Embodiments of oxygen therapy system 5 include athletic performance protocols. Without limitation, athletic performance protocols may maximize endurance, VO₂ max, and recovery. In an embodiment, the athletic performance protocol is of a duration from about 20 minutes to about 30 minutes, alternatively from about 20 minutes to about 25 minutes, further alternatively from about 25 minutes to about 30 minutes, and alternatively from about 20 minutes to about 30 minutes. The athletic performance protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of about 3 minutes of a hypoxic state followed by about 2 of a hyperoxic state. The athletic performance protocol has a hypoxia state oxygen concentration from about 10% to 15%, alternatively from about 12% to about 15%, and alternatively from about 10% to about 12%, and has a hyperoxic state oxygen concentration from about 30% to about 40%, alternatively from about 30% to about 80%, and alternatively from about 30% to about 70%, and from about 40% to about 70%, and from about 40% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the athletic performance protocols are carried out 3 times a week until obtaining the desired result. In the hypoxic state, user 29 may have a target heart rate of from about 75% to about 85% of maximum. The hypoxic state may with oxygen levels lowered simulate a high-altitude environment, which may induce adaptations in the cardiovascular and muscular systems. The hyperoxic state with increased oxygen levels (i.e., oxygen saturation) may promote lactate clearance, enhance tissue oxygenation, and support recovery. In embodiments, athletic performance protocols may include a stationary bike protocol and a treadmill protocol. In embodiments of the stationary bike protocol, the stationary bike protocol may have a warm-up stage which is about 5 minutes at a low resistance level, which may increase the heart rate to about 60% to about 65% of maximum. In the following hypoxic state of reduced oxygen, the reduced oxygen is provided to user 29 for about 3 minutes at about 12% to about 15% oxygen, and is at about a moderate to high resistance level at about 75% to about 80% maximum heart rate. The hypoxic state also has a target saturation of about 85% to about 88%. After the hypoxic state duration is achieved, in the following hyperoxic state of increased oxygen, the increased oxygen is provided to user 29 for about 2 minutes at about 30% to about 40% oxygen, and is at a reduced resistance level at about 70% to about 75% maximum heart rate. The hyperoxic state also has a target saturation of about 95% to about 98%. There are about 4-5 cycles for a total session duration of 20-25 minutes. In embodiments of the treadmill protocol, the treadmill protocol may have a warm-up stage which is about 5 minutes at a slow pace and an incline, which may increase the heart rate to about 60% to about 65% of maximum. In the following hypoxic state of reduced oxygen, the reduced oxygen is provided to user 29 for about 3 minutes at about 12% to about 15% oxygen, and is at a high incline and/or speed at about 80% to about 85% maximum heart rate. The hypoxic state also has a target saturation of about 85% to about 88%. After the hypoxic state duration is achieved, in the following hyperoxic state of increased oxygen, the increased oxygen is provided to user 29 for about 2 minutes at about 30% to about 40% oxygen, and is at a moderate pace with reduced incline at about 70% to about 75% maximum heart rate. The hyperoxic state also has a target saturation of about 95% to about 98%. There are about 4-5 cycles for a total session duration of 20-25 minutes.

Embodiments of oxygen therapy system 5 include respiratory protocols. Without limitation, respiratory protocols may enhance lung capacity, oxygen uptake, and may reduce breathlessness in respiratory conditions. In an embodiment, the respiratory protocol is of a duration from about 15 minutes to about 30 minutes, alternatively from about 15 minutes to about 20 minutes, and alternatively from about 20 minutes to about 30 minutes. The respiratory protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of about 1 to about 2 minutes of a hypoxic state, alternatively about 2 minutes of a hypoxic state followed by about 3 to about 4 minutes of a hyperoxic state, alternatively about 4 minutes of a hyperoxic state, and alternatively about 3 minutes of a hyperoxic state. The respiratory protocol has a hypoxic state oxygen concentration from about 10% to 15%, alternatively from about 12% to about 15%, and alternatively from about 10% to about 12% and further alternatively about 15%, and has a hyperoxic state oxygen concentration from about 35% to about 40%, alternatively from about 35% to about 80%, and alternatively from about 35% to about 70%, and from about 40% to about 70%, and from about 40% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the cardiovascular protocols are carried out 2-3 times a week until obtaining the desired result. The hypoxic state may, with shorter hypoxic exposure, limit the strain in respiratory function while encouraging increased oxygen efficiency. The hyperoxic state may provide an oxygen-rich environment to allow the respiratory system to recover from the hypoxic state.

Embodiments of oxygen therapy system 5 include lactate clearance protocols. Without limitation, lactate clearance protocols may enhance post-exercise recovery by accelerating lactate clearance and reducing muscle fatigue. In an embodiment, the lactate clearance protocol is of a duration of about 10 minutes. The lactate clearance protocol has a hypoxic state duration/hyperoxic state duration (i.e., the hypoxic state has a duration and then the hyperoxic state has a duration, followed by the desired amount of hypoxic state and hyperoxic states of the same duration until the protocol duration has been achieved) of about 2 minutes of a hypoxic state followed by about 3 minutes of a hyperoxic state. The lactate clearance protocol has a hypoxic state oxygen concentration from about 10% to 15%, alternatively from about 12% to about 15%, and alternatively from about 10% to about 12% and further alternatively about 15%, and has a hyperoxic state oxygen concentration from about 35% to about 40%, alternatively from about 35% to about 80%, and alternatively from about 35% to about 70%, and from about 40% to about 70%, and from about 40% to about 80%, alternatively from about 30% to about 40%, and alternatively from about 30% to about 70%, and alternatively from about 30% to about 80%, and alternatively from about 70% to about 80%. The hyperoxic state may act as a recovery period. During the hypoxic state, oxygen levels are reduced to user 29 to simulate an environment with limited oxygen supply. Following hypoxia, the hyperoxic state involves increased oxygen levels for user 29. In embodiments, the lactate clearance protocols are carried out 2-3 times a week until obtaining the desired result. The hypoxic state may enhance oxygen efficiency. The hyperoxic state may maximize oxygen delivery.

Although the present invention and its advantages have been described in detail, it should be understood that various changes, substitutions and alterations may be made herein without departing from the spirit and scope of the invention as defined by the appended claims.

1. An oxygen therapy system, comprising:
an oxygen concentrator;
a valve, wherein concentrated oxygen and exhaust air from the oxygen concentrator are directed to the valve and exit the valve as treatment air, and wherein the treatment air is provided to a user;
a breathing mask, wherein the treatment air is provided to the user via the breathing mask; and
an oxygen therapy protocol, wherein the treatment air is provided to the user according to the oxygen therapy protocol, wherein the oxygen therapy protocol comprises:
a hypoxic state and a hyperoxic state, wherein the hyperoxic state follows the hypoxic state;
wherein the oxygen therapy protocol has an oxygen therapy protocol duration from about 10 minutes to about 30 minutes;
wherein the hypoxic state has a duration from about 2 minutes to about 4 minutes;
wherein the hyperoxic state has a duration from about 1 minute to about 3 minutes;
wherein a hypoxic state oxygen concentration of the treatment air in the hypoxic state is from about 10% to about 15%; and
wherein an hyperoxic state oxygen concentration of the treatment air in the hyperoxic state is from about 30% to about 80%.

2. The system of claim 1, wherein the oxygen therapy protocol comprises more than one hypoxic state and more than one hyperoxic state, and wherein each of the hypoxic states are followed by each of the hyperoxic states in a successive order with a hyperoxic state that follows a hypoxic state in turn followed by a succeeding hypoxic state.

3. The system of claim 2, wherein the successive order is carried out until the oxygen therapy protocol duration has been achieved.

4. The system of claim 1, wherein the oxygen therapy protocol comprises a metabolic disorder protocol.

5. The system of claim 1, wherein the oxygen therapy protocol comprises a mitochondrial protocol.

6. The system of claim 1, wherein the oxygen therapy protocol comprises a weight loss protocol.

7. The system of claim 1, wherein the oxygen therapy protocol comprises a neurological protocol.

8. The system of claim 1, wherein the oxygen therapy protocol comprises a cardiovascular protocol.

9. The system of claim 1, wherein the oxygen therapy protocol comprises an athletic performance protocol.

10. The system of claim 1, wherein the oxygen therapy protocol comprises a respiratory protocol.

11. The system of claim 1, wherein the oxygen therapy protocol comprises a lactate clearance protocol.
